(19) United States
(12) Patent Application Publication
(10) Pub. No.: US 2016/0032327 A1
(43) Pub. Date: Feb. 4, 2016

(54) PROCESS FOR CULTURING DEINOCOCCUS BACTERIA

(71) Applicant: DEINOVE, GRABELS (FR)

(72) Inventors: FABIEN COZE, MONTPELLIER (FR); PATRICK HIVIN, SAINT JEAN DE VEDAS (FR); JEAN-PAUL LEONETTI, MONTPELLIER (FR)

(21) Appl. No.: 14/410,573

(22) PCT Filed: Jul. 1, 2013
(86) PCT No.: PCT/EP2013/063871

§ 371 Date: Dec. 23, 2014

Related U.S. Application Data
(60) Provisional application No. 61/667,060, filed on Jul. 2, 2012.

(30) Foreign Application Priority Data
Jul. 2, 2012 (EP) 12305794.5

(51) Int. Cl. C12P 7/06

(52) U.S. Cl. CPC C12P 7/065

(57) ABSTRACT

The invention relates to processes of culture of a Deinococcus or a related bacterium using an oxidizer in conditions suitable for allowing the growth of Deinococcus and decreasing the growth of at least one other microorganism.

The present invention relates to improved methods for culturing Deinococcus or related bacteria. More specifically, the invention relates to methods for culturing (e.g., growing, fermenting, or maintaining) Deinococcus or related bacteria using an oxidizer, as well as to the use of oxidizers in culture processes or media for favoring the growth of a Deinococcus or a related bacterium among other microorganisms. The invention may be used in fermentation processes, e.g., for producing compounds of interest. The invention further relates to installations suitable for performing fermentation processes, which are optimized by using an oxidizer together with a Deinococcus bacterium.

### Context of the Invention

The use of microorganisms to conduct fermentations, bioproductions or to degrade or modify complex substrates has been proposed in the art.

Fermentation processes may be used for producing substantial quantities of microbial enzymes (e.g. catalase, lipase etc.), metabolites (e.g. ethanol, amino acids, vitamins, etc.), recombinant products (e.g. insulin, interferon etc.), and/or for biotransforming substrates, and the resulting fermented products have applications in several industries, including chemical, pharmaceutical, biotechnological, and food industries. Generally, a pure culture of chosen microorganisms is introduced in a system adapted for conducting the fermentation process, said system further containing a sterilized growth medium.

However, one of the main limitations of fermentation processes remains the presence and the increase of microorganisms that contaminate the cultures.

Currently, heat, irradiations and/or acids/bases are used for decontaminating the installations prior to inoculation of the chosen microorganisms. Nevertheless, such decontaminations are not selective and cannot be performed during the fermentation process itself. Even if the installation and/or the culture medium are sterilized previously, the eradication of contaminants is most often not satisfactory and at least parts of them are inoculated in the installation together with the chosen microorganisms and/or together with the culture medium. In addition, if the fermentation process is continuous, an upstream flux of culture medium feeds the installation. This exogenous contribution may increase the risk of contamination of the fermentation process, since the upstream flux may contains further contaminants.

These contaminations have a major impact on the performances of fermentation processes. The contaminating microorganisms can compete with the chosen microorganisms initially inoculated and/or eradicate them. Most often, it is necessary to interrupt the fermentation process, and to empty out and clean up the installation before loading it again with fresh culture medium and chosen microorganisms.

Sometimes, antibiotics are added to the fermentation tanks, for suppressing part of the contaminants. However, though generally effective, antibiotics have several disadvantages. One of them, besides the cost, is that the antibiotics carry though the fermentation process and end up in the by-products. A second disadvantage of antibiotics is that some bacteria can become resistant over time rendering the use of antibiotics less effective.

Nowadays, these contamination events cannot be avoided for a certainty and they impact on the rate of profit of the installations and on the production cost.

In the recent decades, industrial processes using Deinococcus bacteria for the fermentation and extraction of biofuel, such as ethanol, have been developed. Their ability to ferment biomass and to produce useful metabolites therefrom has participated to the development of improved processes capable of leading to fermentation products that are cheaper and easier to upgrade.

In this regard, Deinococcus bacteria have valuable properties for use in industrial processes or reactions. In particular, WO2009/063079 describes the use of bacteria of the genus Deinococcus for the production of bioenergy products and metabolites from biomass.

WO2010/094665 describes bacteria of the genus Deinococcus having the ability to hydrolyse the main constituents of lignocellulosic biomass, including cellulose, hemi-cellulose (mainly xylan) and lignin, under conditions suitable for an industrial process. WO2010/081899 discloses the ability of Deinococcus bacteria to produce valuable drugs, including antibiotics.

Because such industrial processes often require very large amounts of bacteria, large fermentors, raw substrates, and/or use high sterility conditions, it is important to define cost effective conditions and processes suitable to maintain the growth and activity of the bacteria of interest, as well as a low contamination during the process.

By conducting further experiments and researches on Deinococcus, the inventors have demonstrated that bacteria of the genus Deinococcus exhibit an increased resistance to oxidizers such as hydrogen peroxide as compared to reference bacteria and yeasts, such as Z. mobilis, E. Coli and S. cerevisiae. Furthermore, the inventors have shown that Deinococcus may be cultivated in the presence of an oxidizer under conditions suitable to oxygenate the culture, prevent, reduce or suppress contamination by other microorganisms, favour the growth of Deinococcus and allow optimal biological activity of Deinococcus.

More particularly, Deinococcus can be advantageously used in a process of fermentation, wherein an oxidizer contributes to decrease contamination by less resistant infectious microorganisms. A large part of microorganisms may be eradicated or at least affected using adapted quantities of an oxidizer, such as H₂O₂, which are non lethal for a Deinococcus.

### SUMMARY OF THE INVENTION

One aspect of the present invention therefore relates to a culture of Deinococcus or related bacteria comprising at least one oxidizer.

The invention also relates to a process for culturing Deinococcus or related bacteria, the process comprising culturing said bacteria in the presence of an oxidizer. The oxidizer may be added or supplied at any time during the process, e.g., before, during and/or after addition of the bacteria. The oxidizer may also be repeatedly or continuously supplied or added to the culture.

The invention may be used to cultivate, grow or expand Deinococcus or related bacteria, to preferentially or selectively amplify such cells, to identify or select such cells, in a fermentation or bio-production method, to degrade or transform a biomass, detoxify a substrate or environment and, more generally, in any reaction or process involving the use of Deinococcus or related bacteria.

The invention also relates to a system or installation comprising a fermentor or reactor comprising Deinococcus or related bacteria, and a source of an oxidizer.

The invention further relates to the use of at least one oxidizer in a process of culturing or maintaining or growing Deinococcus or related bacteria.

The invention also relates to a process for sterilizing or decontaminating a culture of Deinococcus or related bacteria, the process comprising exposing said culture to an oxidizer.

The invention also relates to a fermentation process for producing a compound of interest from a culture medium combining the use of a Deinococcus or related bacterium and the use of hydrogen peroxide.

In the present invention, the at least one oxidizer is used preferably under conditions allowing the growth of a Deinococcus or a related bacterium, e.g., below the Inhibitory Concentration of said oxidizer towards said Deinococcus or a related bacterium (“ICD”). Preferably, the oxidizer is used under conditions that inhibit or reduce the growth of at least one other microorganism, such as e.g., E. coli. In a typical embodiment, the oxidizer is used (e.g., added or maintained) at a concentration between 0.05ICD and 0.95ICD, and preferably between 0.25ICD and 0.90ICD.

In this regard, in a particular embodiment, the present invention relates to the use of an oxidizer for selectively decontaminating a culture medium and rescuing a Deinococcus or a related bacterium. The oxidizer acts as a potent antimicrobial agent that promotes the growth of a Deinococcus and reduces the risk of contaminations within the culture medium. Such selective decontamination can be used for selecting a Deinococcus among other microorganisms and/or for improving a fermentation process using a Deinococcus or a related bacterium.

Furthermore, the use of an oxidizer during early stage of a fermentation process (i.e. the degradation step wherein the biomass is degraded into fermentable sugars) may favour oxygenation of the culture medium, thereby reducing exogenous oxygen requirements.

In addition, H2O2 may be used for pre-treating a biomass, such as a lignocellulosic biomass. H2O2 may partially degrade the lignin. This delignification increases the capacity of Deinococcus to use lignocellulosic biomass as a source of carbon, and then improves the fermentation process. In addition, the use of H2O2 instead, at least partially, of heat pre-treatment may advantageously reduce formation of xenobiotic compounds, such as hydroxymethylfurfural and furfural, which inhibit bacterial growth. Since Deinococcus is resistant to H2O2, which may be used for both decontaminating and pre-treating biomass, the combined use of Deinococcus and H2O2 in a microbial production process, such as a fermentation process, is particularly interesting.

Accordingly, in a preferred embodiment the present invention relates to a process of culture of a Deinococcus or related bacterium, wherein at least one oxidizer is used under conditions that allow the growth of a Deinococcus or a related bacterium and decrease the growth of at least one other microorganism.

The oxidizer is preferably chosen among bromine, bromates, chlorinated isocyanides, chlorates, chromates, bichromates, hydroperoxides, hypochlorites, inorganic peroxides, ketone peroxides, nitrates, nitric acid, perborates, perchlorates, perchloric acid, periodates, permanganates, hydrogen peroxide, ozone, peroxides, peroxy acids and persulfates. More preferably, the oxidizer is hydrogen peroxide.

The invention may be used for selecting a Deinococcus or a related bacterium among at least one other microorganism, such as a competitive bacterium (e.g. Z. mobilis, E. Coli), a pathogenic fungus, a yeast (e.g. S. cerevisiae) etc.

The invention may also be used for preparing a fermentation starter containing a Deinococcus or an extract thereof.

The invention may further be used for fermenting a growth medium, preferably in a fermentation system such as a fermentor, a bioreactor or a chemostat.

The process of the invention may further be used for bioconverting a substrate into a substance of interest, such as an aliphatic compound into a dicarboxylic acid.

A further object of the invention relates to a fermentation tank, such as a fermentor, a bioreactor or a chemostat, comprising a growth medium, at least one oxidizer or source of oxidizer and a Deinococcus or a related bacterium or an extract thereof.

A further subject of the invention relates to the use of an oxidizer in a fermentation tank under conditions that allow the growth of a Deinococcus or a related bacterium and decrease the growth of at least one other microorganism.

The invention also relates to a method for reducing bacterial contamination in a fermentor during a microbial production process using a Deinococcus bacterium, wherein the method comprises adding H2O2 to the fermentor.

A further subject of the invention relates to a method of fermentation using a bacterium, wherein the bacterium is a Deinococcus bacterium or related bacterium and the method is conducted in the presence of H2O2.

These and the other objects and embodiments of the invention will become more apparent after the detailed description of the invention.

### LEGEND TO THE FIGURE

FIG. 1: Graphs showing effects of H2O2 on M36-7D_{—}21 growth over time. H2O2 is supplied at the beginning of M36-7D_{—}21 culture or at the exponential growth.

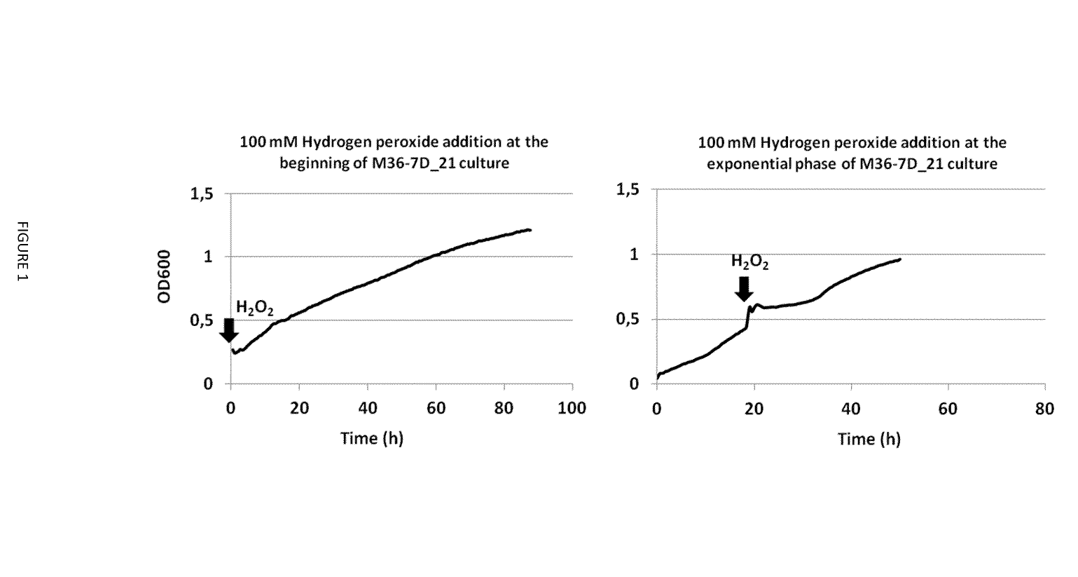

### DETAILED DESCRIPTION OF THE INVENTION

The greater resistance of Deinococcus or related bacteria to oxidizers compared to other microorganisms may advantageously be used for promoting their growth and improving processes using Deinococcus bacteria. Since the use of oxidizers is compatible with both conditions allowing the growth of Deinococcus and the production of fermentation products, adapted quantities of oxidizer(s) can be used, not only as preliminary decontaminant, but as selective decontaminant during the whole fermentation process too.

The following is a description of the present invention, including preferred embodiments thereof given in general terms. The present invention is further exemplified in the disclosure given under the heading “Examples” herein below, which provides experimental data supporting the invention, examples of fermentation processes according to the invention and means of performing the invention.

Definitions

The present disclosure will be best understood by reference to the following definitions.

In the context of the invention, the term “Deinococcus” includes wild type or natural variant strains of Deinococcus, e.g., strains obtained through accelerated evolution, by DNA-shuffling technologies, or recombinant strains obtained by insertion of eukaryotic, prokaryotic and/or synthetic nucleic acid(s). Deinococcus bacteria can designate any bacterium of the genus Deinococcus, such as without limitation, a D. geothermalis, D. cellulolysiticus, D. radiodurans, D. proteolyticus, D. radiopugnans, D. radiophilus, D. grandis, D. indicus, D. frigens, D. saxicola, D. maricopensis, D. marmoris, D. deserti, D. murrayi, D. aerius, D. aerolatus, D. aerophilus, D. aetherius, D. alpinitundrae, D. altitudinis, D. apachensis, D. aquaticus, D. aquatilis, D. aquiradiocola, D. aquivivus, D. caeni, D. claudionis, D. ficus, D. gobiensis, D. hohokamensis, D. hopiensis, D. misasensis, D. navajonensis, D. papagonensis, D. peraridilitoris, D. pimensis, D. piscis, D. radiomollis, D. roseus, D. sonorensis, D. wulumuqiensis, D. xibeiensis, D. xinjiangensis, D. yavapaiensis or D. yunweiensis bacterium. Preferred Deinococcus bacteria are D. geothermalis, D. cellulolysiticus, D. deserti, D. murrayi, and D. radiodurans.

A bacterium “related” to Deinococcus designates a bacterium which (i) contains a 16S rDNA which, upon amplification using primers GTTACCCGGAATCACTGGGCGTA (SEQ ID NO: 1) and GGTATCTACGCATTCCACCGCTA (SEQ ID NO: 2), generates a fragment of about 158 base pairs and/or (ii) resists a UV treatment of 4 mJ/cm². In a particular embodiment, Deinococcus -related bacteria are bacteria having a 16S rDNA molecule which is at least 70%, preferably at least 80% identical in sequence to a Deinococcus 16S rDNA sequence.

An “oxidizer” or “oxidizing agent” refers to a substance that may transfer an oxygen atom to a substrate and/or whose spontaneous degradation produces oxygen. The oxidizer may be in liquid, gaseous and/or solid form. The oxidizer is preferably biocompatible, e.g., suitable for use in a culture process of microorganisms. Advantageously, the oxidizer is hydrogen peroxide.

In the context of the invention, a “growth medium” or “culture medium” designates a medium suitable for supporting the growth of microorganisms such as a Deinococcus or a related bacterium. The growth medium typically comprises at least a source of carbon, a source of amino acids and nitrogen, a source of phosphor. The growth medium can optionally comprise compounds, such as antibiotics that selectively inhibit and/or selectively enhance the growth of specific microorganisms. In particular embodiments, the growth medium can comprise substrates that are required for producing particular compounds, such as substrates used in biotransformation processes. In a particular embodiment, the growth medium comprises or consists of biomass.

The term “biomass” according to the invention typically designates any biological material, from living or recently living organisms. In particular, the term biomass includes unprocessed material of biological origin, including vegetal or animal biomass. Examples of biomass include, without limitation, forestry products, including mature trees unsuitable for lumber or paper production, pulp, recycled paper, organic waste, agricultural products, such as grasses, straw, crops and animal manure, and aquatic products, such as algae and seaweed. Examples of biomass include wood or vegetal material derived from numerous types of plants, including miscanthus, hemp, switchgrass, sugarbeet, wheat, barley, corn, rice, soy, rapeseed (including canola), sorghum, sugarcane, peanut, cotton, lupine, and a variety of tree species, ranging from eucalyptus to oil palm, poplar, willow. Specific sources of biomass include, without limitation, plant residues, hardwood or softwood stems, cobs, straw, grass, leaves, seeds, paper, etc. (see for instance Sun et al., Bioresource Technology 83 (2002) 1-11). The term biomass also encompasses transformed biomass or secondary biomass, which essentially contains hydrolysed pre-treated biomass products. In a preferred embodiment, biomass according to the invention comprises any lignocellulosic material, for example, cellulose, hemicelluloses and/or xylan.

The term “lignocellulosic biomass” according to the invention designates a raw biomass containing lignin, cellulose and/or xylan. The term lignocellulosic biomass thus essentially designates unprocessed material of biological origin. The term lignocellulosic biomass should be distinguished from transformed biomass or secondary biomass, which essentially contains hydrolysed pre-treated biomass products. Examples of lignocellulosic biomass include but not limit to wood or vegetal material derived from numerous types of plants, including miscanthus, rapeseed, switch grass, hemp, sugarbeet, wheat, corn, poplar, willow, sorghum, sugarcane, and a variety of tree species, ranging from eucalyptus to oil palm.

In the context of the invention, the term “process of fermentation” designates any process of production of a product from a carbon source, by means of a microorganism. Fermentation processes include chemical reactions such as oxidations, reductions, polymerizations, and hydrolysis, as well as biosynthesis, and may require the presence or the absence of air. Advantageously, the process of fermentation may be performed in an installation, or fermentation tank, specifically dedicated for producing compounds of interest.

In the context of the invention, the term “biotransformation” or “bioconversion” is the action mediated by a bacterium or an extract thereof for converting a given substrate into a substance of interest. The substrate is distinct from the carbon source and usually not assimilated by the bacterium.

The term “fermentation tank” includes a fermentation system comprising one or more vessels and/or towers or piping arrangements. As is described herein after, in some embodiment, the fermentation tank may comprise a first growth reactor and a second fermentation reactor. As such, when referring to the addition of an oxidizer to the fermentation tank or fermentation reaction, it should be understood to include addition to either or both of these reactors, where appropriate.

In the context of the invention, the “Inhibitory Concentration of an oxidizer towards a microorganism” (“IC”) refers to the minimum concentration of said oxidizer in the culture medium that inhibits or reduces the growth of said microorganism, in such a way that there is no detectable growth of said microorganism after 3 days of culture, preferably after 2 days. And the “Inhibitory Concentration of an oxidizer towards a Deinococcus or a related bacterium” (“ICD”) refers to the minimum concentration of said oxidizer in the culture medium that inhibits or reduces the growth of the Deinococcus. The IC may be easily ascertained for each microorganism or each strain thereof, and for each oxidizer, by implementing test cultures. For example, the strain of interest may be cultivated in several parallel cultures, each of said cultures containing an increased concentration of the oxidizer. After 2 or 3 days, the cultures are observed to measure the microbial growth and thereby to determine the IC.

Culture Process

The inventors have found that the use of at least one oxidizer in a culture medium may contribute to maintain favorable conditions for growth of a Deinococcus or a related bacterium and to reduce the growth of other microorganisms.

The inventors have also found that the presence of an oxidizer may improve Deinococcus culture/fermentation performances. The invention may therefore be used in improved methods of culture, growth, fermentation, and/or isolation/selection of Deinococcus bacteria.

Fermentation Process

As exposed above, microbial contaminations, such as bacterial, viral or fungal contaminations, can make serious damages for fermentation processes, leading to halt the production lines, empty and clean up the installations, before repeating the fermentation processes. Even if preparatory decontaminating procedures are used to sterilize the installation prior to perform fermentation, microbial contaminants may still remain inside the installation and/or microbial contaminants may be provided through the growth medium and/or through the culture medium containing the chosen microorganisms which must be inoculated.

It is therefore an object of the present invention to propose an improved process of fermentation using Deinococcus, wherein an oxidizer is used to decrease or eradicate contamination by infectious microorganisms which are less resistant to said oxidizer. The fermentation processes of the invention may be used for producing substantial quantities of microbial enzymes (e.g. catalase, lipase, etc.), metabolites (e.g. ethanol, amino acids, vitamins, etc.), recombinant products (e.g. insulin, interferon etc.), and/or for biotransforming substrates, and the resulting fermented products have applications in several industries, including chemical, pharmaceutical, biotechnological, and food industries.

In one aspect, the invention provides an improved process of fermentation, suitable for producing several kinds of compounds, by combining the use of a Deinococcus or a related bacterium, and the use of an oxidizer, more particularly the use of H2O2. More particularly, the invention provides an improved process of fermentation, for producing compounds of interest from a biomass by combining the use of a Deinococcus or a related bacterium, and the use of H2O2.

H2O2 may be particularly useful during a process of fermentation. Indeed, the addition of H2O2 is compatible with conditions allowing both the growth of Deinococcus and production of fermentation products. Appropriate doses of H2O2 can be used not only as preliminary decontaminant but as selective decontaminant during the whole fermentation process too.

Such a process of fermentation can be performed without antibiotic or with a reduced amount of antibiotics compared to current fermentation processes. In one embodiment, the oxidizer may be used instead of antibiotics for decreasing the amount of contaminant microorganisms during a fermentation process.

In a particular embodiment, the process of fermentation comprises the steps of:

a) contacting a growth medium comprising a source of carbon with an oxidizer,

b) contacting the growth medium with a Deinococcus, or a related bacterium, in conditions suitable for culturing and/or growing said bacterium ;

c) culturing Deinococcus for producing compounds of interest, and optionally

d) collecting the compounds of interest resulting from said fermentation.

According to the invention, steps a) and b) may be performed simultaneously or sequentially, depending on the culture medium, the volume of culture, the compounds etc.

The process of fermentation of the present invention may be implemented for producing a wide range of products. Those skilled in the art may easily adapt the conditions to favour the production of products of interest, by adjusting the composition/nature of the culture medium, the amount of oxidizer and/or of Deinococcus etc., to recover optimum amounts of such products of interest.

In particular embodiments, the process of fermentation is used for the production of metabolic products, such as amino acids, proteins (including enzymes), vitamins, alcohols etc., for human and/or animal consumption or industrial use, modification of compounds through biotransformation, production of recombinant products, and production of microorganisms themselves (for use as animal feed for example).

According to the invention, the culture medium can contain biomass.

In particular embodiments, the culture medium can further contain one or more substances (e.g. amino acids, vitamins or mineral salts) that favor the growth of Deinococcus and/or the production/bioconversion of compounds of interest.

The optimized conditions to favor the growth of the Deinococcus bacteria are set by those skilled in the art. In the same way, those skilled in the art know substrates and/or source of carbon required for producing a given compound. Examples of process of fermentation that may be easily adapted by those skilled in the art are disclosed in WO02009/063079 relative to the production of bioenergy products, and/or in WO02010/081899 relative to the production of pharmaceutical compounds, and/or in WO02010/094665 relative to the production of fermentable sugars.

Selection Process

In a further aspect, the invention provides a method of selectively culturing a Deinococcus from a sample containing several microorganisms.

Indeed, the higher resistance of bacteria of the Deinococcus genus to oxidizers, such as peroxygen compounds (e.g. H₂O₂), compared to other microorganisms, including bacteria, fungi, viruses, can be useful for isolating/selecting Deinococcus bacteria among other uncharacterized microorganisms.

In one embodiment, a sample comprising uncharacterized microorganisms is subjected to an oxidizer under suitable conditions for allowing the growth of a Deinococcus, or a related bacterium and decreasing the growth of at least one other microorganism. Then, the living or growing microorganisms may be isolated from the treated sample and a Deinococcus or a related bacterium further selected.

In particular embodiments further parameters of culture may be adjusted to fulfill the selection. For example, the pH of the culture may be kept between 4 and 10 and/or the temperature of the culture may be kept between 4° C. and 70° C.

Those skilled in the art may easily adapt the conditions of culture to the processing specificities.

In another aspect, the invention provides a method of preparing a fermentation starter, containing a Deinococcus or a related bacterium or an extract thereof. Starters are generally used for initiating and/or assisting at least the beginning of a fermentation process.

The fermentation starter of the invention advantageously comprises a culture of

Deinococcus or related bacteria, or an extract thereof, among other ingredients. For example, after the culture and/or the selection of the Deinococcus or related bacteria, the bacteria are transformed into a paste with an adapted culture medium and optionally additional ingredients such as exogenous enzymes, antibiotics etc.

The fermentation starter of the invention can be used in several fermentation processes, and more particularly in fermentation processes for degrading biomass. Advantageously, a suitable amount of the fermentation starter is mixed with the biomass for starting the fermentation process. The amount of fermentation starter can be easily adapted, depending e.g., on the biomass and/or the products of fermentation.

Oxidizer Supply in a Process of the Invention

According to the invention, the oxidizer may be added once, continuously or periodically, depending on the process of fermentation. And, the concentration of the oxidizer can be controlled or monitored during all the process, using suitable sensors. The concentration may be adjusted e.g., by adding further amounts of oxidizer, or conversely, further diluents and/or fresh culture medium and/or fresh microorganisms.

The process of fermentation of the invention may be adapted for all kinds of fermentation processes, including batch processing, fed batch processing, and continuous processing. Those skilled in the art may easily adapt the conditions of culture to the processing specificities.

In one embodiment, additional quantities of oxidizer are added into the fermentation tank at once adding a fresh medium, so that the concentration of oxidizer remains suitable for affecting the growth of microbial contaminants without inhibiting the growth of the Deinococcus.

For example, when the fermentation process is performed using a batch processing, a single dose of oxidizer may be used each time a new batch is provided, preferably mixed with the new batch.

When the fermentation process is performed using a continuous processing, wherein fresh medium is continuously added to a chemostat, the oxidizer may be added continuously too, preferably together with the fresh culture medium.

In another embodiment, the concentration of the oxidizer is regularly checked and adapted amounts of oxidizer are added if required.

In one embodiment, two or more chemostats may be connected in series, the overflow of the previous chemostat being recovered in the following chemostat. Advantageously, each chemostat of the system is filled with doses of oxidizer. Otherwise, or in addition, the oxidizer can be supplied together with the overflow, for decreasing the risk of cross-contamination between the chemostats.

In a particular embodiment, the oxidizer is used only during the degradation step of the process. Advantageously, no further oxidizer is used during the fermentation step itself. During the degradation step, the oxygen requirement is advantageously fulfilled by supplying the oxidizer in the culture medium, which produces oxygen inside the culture. For example, hydrogen peroxide may be used as an oxidizer during the degradation step. Hydrogen peroxide decontaminates the culture and decomposes into water and oxygen spontaneously. Thanks to this endogenous production of oxygen inside the culture, no further exogenous oxygen is required. During the second part of the process (i.e. the fermentation step), no oxidizer is further added. Since the oxidizer has been decomposed previously, the culture is free of both oxidizer and oxygen. The fermentation step may be implemented, anaerobic conditions being fulfilled.

In a further embodiment, a mixture of oxidizer and microorganisms is inoculated in a fermentation tank before, simultaneously or after the filling of said fermentation tank with the culture medium. The oxidizer acts as a selecting agent for eliminating non resistant microorganisms from the mix of microorganisms, and that way selecting at least one Deinococcus or related bacterium. For example, in order to inhibit the growth of a large part of microorganisms other than Deinococcus, H₂O₂ may be used up to 200 mM, and preferably up to 180 mM.

In addition, by changing the culture conditions (e.g. temperature, pH, concentrations, reaction time, presence/absence of catalysers, etc.) the selective lethal effects of the oxidizer can be increased. For example, exponentially growing bacteria will be more sensitive to H₂O₂ than non growing bacteria.

In the same way, helpers may be used together with the oxidizer for focusing their lethal properties on chosen microorganisms. For example, by combining H₂O₂ and ascorbic acid, it is possible to eradicate gram-negative bacteria selectively. Otherwise, or in addition, two or more oxidizers may be used simultaneously.

In the present invention, the at least one oxidizer is used preferably under conditions allowing the growth of a Deinococcus or a related bacterium, e.g., below the ICD. Preferably, the at least one oxidizer is used under conditions that inhibit or reduce the growth of at least one other microorganism, such as E. coli.

In a typical embodiment, the at least one oxidizer is used (e.g., added or maintained) at a concentration between 0.05ICD and 0.95ICD (i.e. between 5% and 95% of the ICD), for instance up to 0.1ICD, 0.2ICD, 0.3ICD, 0.4ICD, 0.5ICD, 0.6ICD, 0.7ICD, 0.8ICD, or 0.90ICD. The concentration may be adjusted by the skilled person depending on the bacterium used, the process conditions, the nature of the oxidizer and/or the culture system.

As an example, H₂O₂ may be used or maintained at a concentration of between 10 mM and 300 mM, preferably between 30 mM and 180 mM in order to eliminate at least E. Coli, S. Cerevisiae and/or Z. mobilis.

Pre-Treatment of a Lignocellulosic Biomass

The conversion of lignocellulosic biomass has been the subject of intense research efforts since the 1970s (Blumer-Schuette et al., 2008, “Extremely thermophilic microorganisms for biomass conversion: status and prospects”, Curr Opinion Biotechnol 19, pp. 210-217; Perez et al., 2002, Int Microbiol 5, pp 53-63). As reported in Mosier et al. (Bioresource Technology 96 (2005) 673-686), the pre-treatment of lignocellulosic biomass is required to alter the structure of cellulosic biomass to make cellulose more accessible to the enzymes that convert the carbohydrate polymers into fermentable sugars.

In this context, the process of the invention proposes to use an oxidizer for pre-treating lignocellulosic biomass by partially degrading the lignin. This delignification increases the capacity of Deinococcus to use lignocellulosic biomass as a source of carbon, and then improves the fermentation process.

For example, the lignocellulosic biomass may be pre-treated with an oxidizer in a concentration range between 1 mM and 1M allowing a partial digestion or solubilisation of the lignin.

This pre-treatment step can be performed during a period of time sufficient for degrading at least 10% of the lignin, and preferentially between 20% and 80%. Then, if required and before the inoculation of bacteria, the resulting mixture may be diluted, by addition of water or other suitable diluents, to reduce the concentration of the oxidizer in a range allowing the growth of a Deinococcus bacterium.

Fermentation Tank

It is an object of the invention to provide fermentation tanks containing means dedicated for implementing the process of fermentation of the invention.

According to the invention, the process of fermentation can be carried out in a fermentation tank, whose capacity/volume may vary from few litters to thousands litters.

Advantageously, the fermentation tank comprises dedicated means, such as injection means, adapted to deliver required quantities of an oxidizer into the fermentation tank.

In a particular embodiment, the fermentation tank comprises a sparger (e.g. “Ring sparger UniVessel®” of Sartorius) for delivering and diffusing the oxidizer into the fermentation tank.

In a particular embodiment, the oxidizer may be produced directly on the fermentation site. Such a production on the fermentation site may be useful for avoiding the transport and the storage of unstable oxidizers. For instance, H₂O₂ may be produced by direct synthesis from hydrogen and oxygen, as described by Rusty Pittman et al. (“On site production of Hydrogen peroxide”, UOP LLC-2003).

In addition, means for conveying the oxidizer into the vessel, such as pipes, may further be provided. For example, the pipes may be dedicated pipes only transporting the oxidizer. Otherwise, the pipes may be common pipes, used to convey, alternatively or simultaneously, culture medium and/or microorganisms and oxidizer into the fermentation tank.

The fermentation tank can further comprise a heat exchanger or thermostats for keeping the temperature constant. More particularly, refrigeration means may be provided for balancing the increase in temperature resulting from the decomposition of the oxidizer into oxygen.

In a particular embodiment, the fermentation tank is free of aerator, the oxidizer supplying the required oxygen.

In one embodiment, the fermentation tank comprises sensors and/or control system for measuring/controlling the concentration of the oxidizer during the process of fermentation.

More generally, those skilled in the art may easily adapt the design of the fermentation tank to the process of fermentation implemented.

### EXAMPLES

Example 1: Determination of the Inhibitory Concentrations (IC) of hydrogen peroxide for wild type and recombinant Deinococcus bacteria

Protocol

Reference strains

Zymomonas mobilis DSM424, 30° C.

Escherichia coli K12, 30° C.

Saccharomyces cerevisiae 2640, 30° C.

Strains to be Tested

Deinococcus radiodurans R1, 30° C.

M36-7Dwt, 45° C.

Preculture

Preculture was done for 3 days in CMG 1%. After checking the purity of the culture, cell pellet was washed three times in sterile water and then DO was adjusted to 2.

Hydrogen Peroxide

Hydrogen peroxide stock solution: H₂O₂ (30%wt solution, SIGMA ref. 216763, stored at 4° C.)

Concentrations tested: 0, 50, 100, 120, 140, 160, 180, 200, 300 and 400 mM

Solution n°1; 1M: 2.3 mL of H₂O₂ stock solution in 20 mL CMG medium.

Solution n°2; 100 mM: 340 μL of H₂O₂ stock solution in 30 mL CMG medium

Preparation of the H₂O₂ range:

| | 5 mL final volume | | |
|---|---|---|---|
| Concentration (mM) | Solution 1 (ml) | Solution 2 (ml) | CMG (ml) |
| 500 | 2.5 | — | 2.5 |
| 400 | 2 | — | 3 |
| 300 | 1.5 | — | 3.5 |
| 200 | 1 | — | 4 |
| 100 | — | 5 | 0 |
| 80 | — | 4 | 1 |
| 60 | — | 3 | 2 |
| 40 | — | 2 | 3 |
| 30 | — | 1.5 | 3.5 |
| 20 | — | 1 | 4 |
| 10 | — | 0.5 | 4.5 |
| 0 | — | — | 5 |

Preparation of the Microplate

Dispense 180 μl of media into culture wells (200 μl into blank wells)

Add 20 μL of washed cell pellet at an optical density (600 nm) of 2 into well to be inoculated.

Gently add 50 μL of mineral oil on the well surface to limit evaporation.

Microplate was incubated for 3 days at 30 or 45° C. according to the optimal growth temperature indicated in the first part. Growth was monitored by an absorbance measure.

Results

The results are summarized in the following table.

| | | | | D. radiodurans | M36- | M36- | M36- | M36- |
|---|---|---|---|---|---|---|---|---|
| | Z. mobilis | E. coli | S. cerevisiae | R1 | 7Dwt | 7D_17 | 7D_18 | 7D_21 |
| IC (mM) | 10 | 10 | 20 | 200 | 200 | 200 | 200 | 200 |

IC: concentration of hydrogen peroxide from which there was no growth after 3 days of incubation.

Compared to reference microorganisms (Z. mobilis, E. coli and S. cerevisiae), D. radiodurans R1 and D. geothermalis M36-7Dwt and modified ones are able to grow in the presence of high concentration of hydrogen peroxide (up to 190 mM).

### Example 2

### Determination of the Inhibitory Concentrations (IC) of Hydrogen Peroxide for Two Recombinant Deinococcus Bacteria at Different Temperatures

Protocol

Deinococcus geothermalis M36-7D_{—}21 and MX61E_{—}04 were cultivated in 20% starch effluent pH 5 containing 15 mM NH₄Cl and 5.30 mM K₂HPO₄

5 ml of the cultures were treated during one hour with different concentration of H₂O₂ (20, 50,100, 150 and 200 mM) at room temperature or at 45° C.

The control sample corresponds to untreated sample (0 mM H₂O₂).

The control and treated samples were then spread on agar plates containing 10% effluent starch and incubated during 24 and 96 hours at 45° C.

The inhibitory concentration (IC expressed in mM) is the hydrogen peroxide concentration from which there was no growth after 24 or 96 h.

Results:

TABLE
Inhibitory concentration (mM) of hydrogen peroxide at
two different temperatures of incubation

| | M36-7D_21 | MX6-1E_04 |
|---|---|---|
| Room temperature | >200 mM | 100 mM |
| 45° C. | 200 mM | 50 mM |

### CONCLUSION

The both strains of D. geothermalis showed good resistance to hydrogen peroxide compared to reference microorganisms (as showed in example 1). In addition, the resistance of D. geothermalis to hydrogen peroxide was higher when the cultures were incubated at room temperature.

### Example 3

### Effect of Hydrogen Peroxide Addition on D. geothermalis Growth Protocol

The growth of Deinococcus geothermalis M36-7D_{—}21 was performed in microplates in CMG medium containing Peptone 2 g/L ; Yeast Extract 5 g/L ; Glucose 55 mM (10 g/L) ; MOPS acid 40 mM; NH₄Cl 20 mM ; NaOH 10 mM; KOH 10 mM; CaCl₂.2H₂O 0.5 μM; Na₂SO4.10H₂O 0.276 mM; MgCl₂.6H₂O 0.528 mM; (NH₄)₆(Mo₇)O₂₄.4H₂O 3 nM; H₃BO₃ 0.4 μM; CoCl₂.6H₂O 30 nM; CuSO₄.5H₂O 10 nM; MnCl₂ 0.25 μM; ZnSO₄.7H₂O 10 nM; D-Biotin 1 μg/L; Niacin (nicotinic acid) 1 μg/L; Pyridoxin (pyridoxal HCl ou vitamine B6) 1 μg/L; Thiamin HCl (vitamine B1); FeCl₃ 20 μM; Sodium Citrate.2H₂O 20 μM; K₂HPO₄ 5,7 mM.

The addition of 100 mM hydrogen peroxide was made at TO (begin of the growth) or at the exponential phase.

The growth was monitored by measuring the OD_{600 nm}.

Results:

The addition of hydrogen peroxide did not affect the growth of M36-7D_{—}21 whatever it was added at TO or at the exponential growth phase.

### Example 4

### Deinococcus Cultivation in Bioreactor with H₂O₂

In the following experiments, a recombinant Deinococcus geothermalis expressing the pyruvate decarboxylase gene and the alcohol dehydrogenase gene from Zymomonas mobilis is used.

4.1- Production of Ethanol Without Pretreatment of the Biomass

1 g dry weight/L of the strain is cultivated on 40 g/L dry wheat in 1 L-bioreactor (Biostat Q+Sartorius). The temperature is kept at 40° C., and the pH is kept at 8.

H₂O₂ is used to fulfill both the oxygen requirement for the growth of the Deinococcus bacteria and the ethanol biosynthesis. For this aim, a solution of H₂O₂ 3M is added continuously to the bioreactor with a pump delivering 0.0015 vvm (oxidizer volume per cultivation volume per minute), so that the concentration of H₂O₂ into the bioreactor is maintained between 170 mM and 180 mM.

A sustained production of ethanol over several days is obtained.

4.2—Production of Ethanol with Pretreatment of the Biomass

Pre-treatment step: A solution of H₂O₂ 5M is added continuously in 1 L-bioreactor (Biostat Q +Sartorius) dry wheat, with a pump delivering 0.0025 vvm during 24 hours.

Then, pure H₂O is added in the bioreactor to reduce the concentration of H₂O₂.

Fermentation step: The concentration of H₂O₂ in the bioreactor is monitored with a sensor, and 2 g dry weight/L of Deinococcus are supplied in the bioreactor when the concentration of H₂O₂ is below 150 mM. The temperature is kept at 55 ° C., and the pH is kept at 7.

In order to maintain a concentration of H₂O₂ into the bioreactor between 170 and 180 mM, a solution of H₂O₂ 2M is added continuously to the bioreactor with a pump delivering 0.0025 vvm.

A sustained production of ethanol over several days is obtained, showing that pre-treatment by the oxidizer increases the lignin degradation and also enhances the ethanol biosynthesis.

### Example 5

### Industrial Process for Deinococcus Cultivation using Hydrogen Peroxide to Decontaminate and Oxygenate the Culture

Deinococcus geothermalis is cultivated on 40 g/L dry wheat in 1 L-bioreactor (Biostat Q+Sartorius). The temperature is maintained at 45° C., and pH is maintained at 6. The H₂O₂ can be used to fulfill the oxygen requirement for the growth. For this aim, a concentrated solution of H₂O₂ in a range of 1 to 5 M is added continuously to the bioreactor with a pump delivering between 2.5 to 0.15 mL H₂O₂ per minute.

1-14. (canceled)

15. A fermentation process for producing a compound of interest from a culture medium comprising combining a Deinococcus or related bacterium and culture medium containing hydrogen peroxide (H2O2).

16. The process of claim 15 comprising the steps of:
a) contacting a culture medium comprising a source of carbon with hydrogen peroxide (H2O2);
b) contacting the culture medium with a Deinococcus, or a related bacterium, in conditions suitable for culturing and/or growing said bacterium;
c) culturing Deinococcus for producing a compound of interest; and optionally
d) collecting the compound of interest resulting from said fermentation.

17. The process of claim 15, wherein the concentration of H2O2 in the culture is between 5% and 95% of the Inhibitory Concentration of H2O2 for said Deinococcus or related bacterium (ICD).

18. The process of claim 15, wherein H2O2 is added or supplied to the culture before and/or at the same time and/or after providing the Deinococcus or related bacterium to the culture.

19. The process of claim 15, wherein H2O2 is added continuously or sequentially to the culture.

20. The process of claim 17, wherein the concentration of H2O2 is monitored and maintained below 95% of the ICD.

21. The process of claim 15, wherein the culture medium comprises a biomass.

22. The process of claim 15, wherein the compound of interest is an alcohol.

23. The process of claim 15, wherein the compound of interest is a fermentable sugar, a metabolite and/or a drug of interest.

24. A fermentation tank comprising a growth medium, H2O2 or a source of H2O2, a Deinococcus or a related bacterium or an extract thereof, and means for conveying H2O2 into the fermentation tank and/or means for producing H2O2 and/or means for monitoring the concentration of H2O2.

25. A method for reducing bacterial contamination in a fermentor during a microbial production process using a Deinococcus bacterium or related bacterium the method comprising adding H2O2 to the fermentor during said microbial production process.


SEQUENCE LISTING (2 sequences)

SEQ ID NO: 1 - DNA, 23 bp, artificial
  primer
gttacccggaatcactgggcgta

SEQ ID NO: 2 - DNA, 23 bp, artificial
  primer
ggtatctacgcattccaccgcta